(19) United States
(12) Patent Application Publication
(10) Pub. No.: US 2012/0219518 A1
(43) Pub. Date: Aug. 30, 2012

(54) Healing House of Isis Hair and Skin Formulation

(76) Inventor: Gina Serra, West Palm Beach, FL (US)

(21) Appl. No.: 13/033,629

(22) Filed: Feb. 24, 2011

(51) Int. Cl. A61K 8/97; A61Q 19/00; A61Q 5/02; A61Q 5/12; A61K 8/92; A61Q 5/00

(52) U.S. Cl. 424/74; 424/70.1

(57) ABSTRACT

The purpose of this product is to provide the consumer with an all natural alternative to other products currently on the market free from alcohol, petroleum and water. The compositions can be formulated as hair and skin products and all contain a percentage of shea butter according to the optimal amount for that product.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Current U.S. Class: 424/725; 424/539; 424/736; 424/745; 424/761; 424/766; 424/770; 424/777; 510/139

### REFERENCES CITED

### Referenced by Patent Number

U.S. Patent Documents

| | | |
|---|---|---|
| 7,186,852 | March 2007 | Rogers et al. |
| 4,737,360 | April 1988 | Allen et al. |
| 5,350,774 | September 1994 | Palou |
| 5,916,573 | June 1999 | Spiers et al. |
| 6,572,868 | June 2003 | Cope |
| 7,700,530 | April 2010 | Mundschau et al. |

### BRIEF SUMMARY OF THE INVENTION

The invention is titled “The Healing House of Isis Skin and Hair products” The products are a unique blend of natural ingredients. The ingredients include 100% organic shea butter, and a blend of essential oils, olive and castor oils and other natural ingredients including tea tree oil, rosemary oil, wild chamomile, clary sage, ylang ylang, oleoresin of frankincense, myrrh, mandarin, geranium, peppermint, rosemary, cedarwood, castile soap, vegetable glycerin, and sugar cane.

The products contain no mineral oil, petroleum or water. The products are blended to make a consistency pleasing to the client.

### BRIEF DESCRIPTION OF THE DRAWINGS

The drawings (1/13 through 13/13) display and help show the percentages of ingredients included in each of the formulations. The totals are shown both in percentages at the bottom of each drawing and in a bar chart format that visually represents the makeup of each ingredient in each formulation.

### DETAILED DESCRIPTION OF THE INVENTION

Shea Butter: For hair it maybe used to help with the regeneration of broken damaged hairlines. It can help to control frizzy hair as well as soften the curl pattern naturally found in coarse hair. As a pressing tool it can be used before applying heat to protect against heat damage through the natural adhering of the shea butter and olive and castor oils to the hair shaft. Can be used to impart moisture on a dry scalp makes an excellent daily grooming for hair as it leaves no residue and is easily absorbed by the skin.

Conditioner: The conditioner nourishes and restructures dry hair from roots to ends. The formula will regenerate and repair dry ends and protect hair against harsh elements (sun, wind, cold, over processing). Conditioning agents shield the capillary fibers and detangle hair leaving the hair shiny, soft and smooth.

Skin Cream: The cream absorbs quickly into the skin to leave skin feeling soft and looking radiant all day. The cream will nourish, moisturize and protect skin.

Shampoo: Protects and restructures hair, adding brilliance and shine. Hair fibers are smoothed and the natural oil balance of the scalp is protected.

Body Wash: The product cleanses the body while preserving the natural skin balance. Leaves the skin soft and supple.

Exfoliator: Formulated with natural sugar crystals to gently exfoliate and cleanses to leaving skin smooth, bright and moisturized, without over-drying.

The present invention relates to novel compounds prepared by the reaction with shea butter, preferably mild-processed shea butter. These compounds are useful as cosmetic and personal care ingredients, allowing for the delivery of highly desirable active ingredients present in shea butter, including natural antioxidants, in compounds are substantive to the skin and hair.

The shea butter used in the products is added to as follows:

Sanitize hands and work area before beginning the process. All ingredients are added with sterilized funnels, liquid eye droppers, and professional mixers. In mixing bowl add enough cold pressed olive oil to moisten applicable shea butter mixture. Adjust speed of mixer cream and blend mixture together adding castor oil and oil blends. Cream until mixture is a smooth consistency. Add sugarcane when required after the smooth consistency of the blend has been achieved.

The product is used on wet hair after shampooing applied sparingly from root to end. No heat is necessary nor is rinsing required. Hair will be soft, smooth and easy to comb thru and the hair is then ready to be styled.

The product has benefits for all ethnic hair types. When applied to natural African hair (hair that is not chemically altered with relaxer or texturizer) along with heat through a series of treatments with the Product, natural hair can then be worn straight just like relaxed hair but without the worry of breakage, scalp burns or the allergic scalp reactions most women experience with relaxers and other chemicals. The health and integrity of the hair is supported by the synergy created by the Product's ingredients. This allows hair growth, and regeneration of broken hairlines.

When used on Caucasian hair, the product will give blond hair a radiant and lasting shine and does not make it heavy or oily. In fact, the Product is very easily absorbed into the hair shaft leaving a wonderfully smooth and polished cuticle removing the frizzies. Using the Product before flat ironing provides heat protection whilst blocking moisture out allowing hair to straighten longer even in humid weather.

People who have mixed race hair and use the product will keep their hair wave straight because there is no water, alcohol, mineral oil or petroleum in the Product. There are also no synthetic perfume scents in the product making it more user friendly. The Product is an excellent hair detangler which makes it completely child friendly. When used in a massage, the product will provide relaxation for the muscles and has great omega nutrients for the skin assisting the skin with vitality and elasticity whilst locking in moisture and with not alcohol in the mix. The skin remains moisture balanced until the next shower.

When used as a conditioner, skin cream, body wash, or exfoliator the shea butter maximizes the effects for the consumer per the chemistry of each particular product.

### Shea Butter

Shea butter extract is a complex fat that contains many non-saponifiable components (substances that cannot be fully converted into soap by treatment with alkali) oleic acid (40-60%); stearic acid (20-50%); linoleic acid (3-11%); palmitic acid (2-9%); linolenic acid (<1%); arachidic acid (<1%).

As it is full of minerals, vitamins, moisturizing and anti-inflammatory properties, shea butter's benefits for hair are many. Its anti-inflammatory properties heal damaged scalp and help clear any infections. It works well against dandruff too. It can do wonders for hair that has been damaged by excessive coloring and blow-drying. It also helps to de-stress hair that has been affected for too long by pollution. Damaged and brittle hair can be conditioned and revitalized by its moisturizing properties. It soothes and softens dry hair. As shea butter is light and non-greasy, it can also be applied as a leave-in for hair. This will help to shield hair by locking or concealing the moisture in. Besides being great for dry hair, it helps to nourish normal hair and it's non-greasy, easy spread property helps to control excess moisture in oily hair. Using shea butter for hair growth is not unknown, due to its rejuvenating minerals and vitamins.

### Clary Sage: Salvia Sclarea

General: The essential oil lends strength, both psychological and physical. While it helps reduce deep-seated tension, it remains stimulating, regenerative, and revitalizing.

### Chamomile: Matricaria Chamomile

An infusion of Chamomile flowers is used as a hair shampoo, especially for fair hair.

The chief constituent of Chamaemelum have been identified as esters of angelic and tiglic, together with amyl and isobutyl alcohol's. It also contains anthemol and a hydrocarbon anthemene. The flowers contain various volatile oils including proazulenes. Upon steam distillation these proazulenes produce chamazulene, this is remarkably anti-allergenic.

### Rosemary: Rosmarinus Officinalis

It is employed principally, externally, as spiritus Rosmarini, in hair-lotions, for its odor and effect in stimulating the hair-bulbs to renewed activity and preventing premature baldness.

### Castile Soap

The castile soap used is made from olive oil, a monounsaturated fatty acid that helps the body manufacture prostaglandins. This helps moisten the skin and improves texture.

The benefits of antioxidants are widely touted. The olive oil in castile soap is a good source of antioxidants. Regular use of castile soap may aid in fighting cell degeneration.

One of the most important constituents of Olive oil is Oleic acid in a percentage of approx 60%-80%. What this means is that olive oil is a skin regenerator and a natural humectant, drawing moisture from the air, keeping the skin soft and supple. The resultant soap that Olive oil produces when saponified is very mild and gentle. Olive oil also contains Squalane which has the ability to help the skin to retain moisture while nourishing and providing protection for all skin types, especially dry and sensitive. It is non-greasy and non-comedogenic, which means that it doesn't restrict or clog the pores in any way. Olive squalene assists the skin's ability to naturally regenerate and feed itself. It is similar in structure to the skin's own squalene composition and because of this it is highly penetrative.

### Ylang-Ylang Oil

Our ylang-ylang oil is extracted from Cananga odorata var. genuina (Unona odorantissimum) of the Anonaceae family and is also known as “flower of flowers” and is from the very first pressing—also called ‘complete’ or ‘extra’.

The main chemical components are linalool, geranyl acetate, caryophyllene, p-cresyl methyl ether, methyl benzoate, benzyl acetate, benzyl benzoate and other sesquiterpenes. There are different ‘pressings’ of ylang-ylang oil—with them being called ‘extra’ and then 1st, 2nd and 3rd pressing. The extra is the very first pressing and contains the highest amounts of esters and therefore has the sweetest odor, while the later pressings have a less-sweet odor.

The ‘extra’ is not used in aromatherapy, but used in the perfume industry and to have a ‘complete’ essential oil for aromatherapy, the ‘extra’ is combined with the 1st and 2nd pressings to form the complete ylang-ylang essential oil.

Ylang-ylang oil is extracted from the freshly picked flowers by water or steam distillation.

The first distillation is called Ylang-ylang extra, which is the top grade. An absolute and concrete are also produced by solvent extraction.

### Sugarcane

Sugarcane is any of 6 to 37 species (depending on which taxonomic system is used) of tall perennial grasses of the genus Saccharum (family Poaceae, tribe Andropogoneae). Native to warm temperate to tropical regions of Asia, they have stout, jointed, fibrous stalks that are rich in sugar, and measure two to six meters (six to nineteen feet) tall. All sugar cane species interbreed, and the major commercial cultivars are complex hybrids.

Glycolic is a naturally-occurring, non-toxic alpha hydroxy acid found in sugar cane. Its small molecular structure facilitates a more effective penetration of skin cells when compared to other alpha hydroxy acids. Glycolic accelerates the removal of dead skin, increases collagen production, and reduces the appearance of fine lines, sun damage and hyper-pigmentation. This exceptional 15% concentration serum also contains apple source malic acid, salicylic acid and organic extracts that diminish large pores, acne scarring, and help clear oily or acne-prone skin.

### Vegetable Glycerin

Vegetable glycerin is also known as vegetable glycerol. It is a carbohydrate that is usually derived from plant oils. Vegetable glycerin is also used in place of alcohol to extract botanicals.

Glycerin is an organic compound composed of three carbon atoms, hydrogen atoms, and three OH groups. These OH groups form hydrogen bonds with water, slowing down its movement and giving liquid glycerin the property of a syrup. It is also resistant to freezing, a property used in storing sensitive liquids, such as enzymes, in laboratory freezers.

1. A hair and skin formulation, comprising shea butter, olive oil, and castor oil.

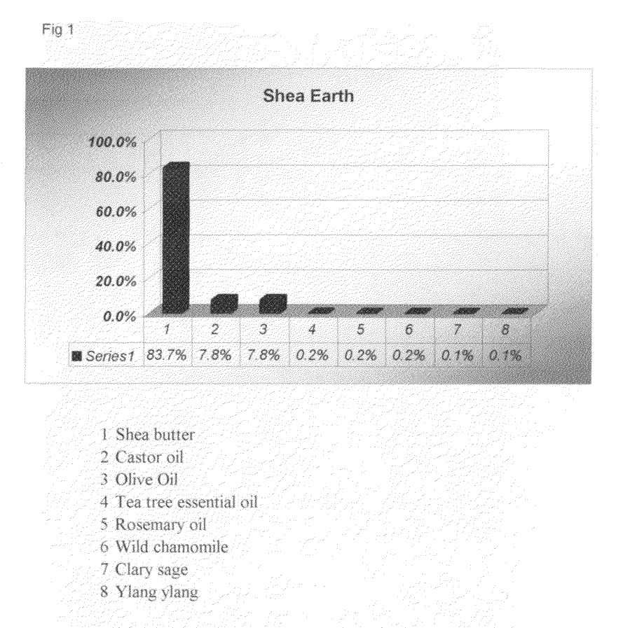

2. The hair and skin formulation according to claim 1 wherein said shea butter is about 84%, olive oil 8%, castor oil 7%, and further comprising about 2% tea tree essential oil, about 0.2% rosemary oil, about 0.2% wild chamomile, about 0.2% clary sage and about 0.2% of ylang ylang. (see FIG. 1).

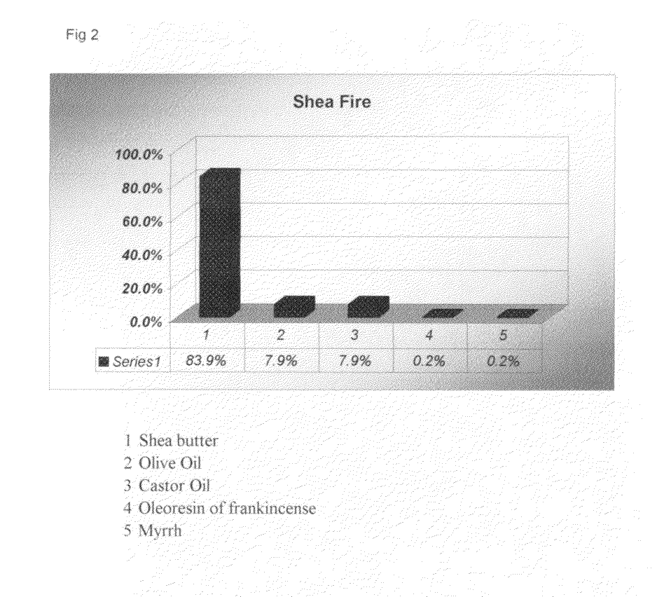

3. The hair and skin formulation according to claim 1 wherein said shea butter is about 83%, olive oil 8%, castor oil 7%, and further comprising about 1% oleoresin of frankincense and 1% myrrh. (see FIG. 2).

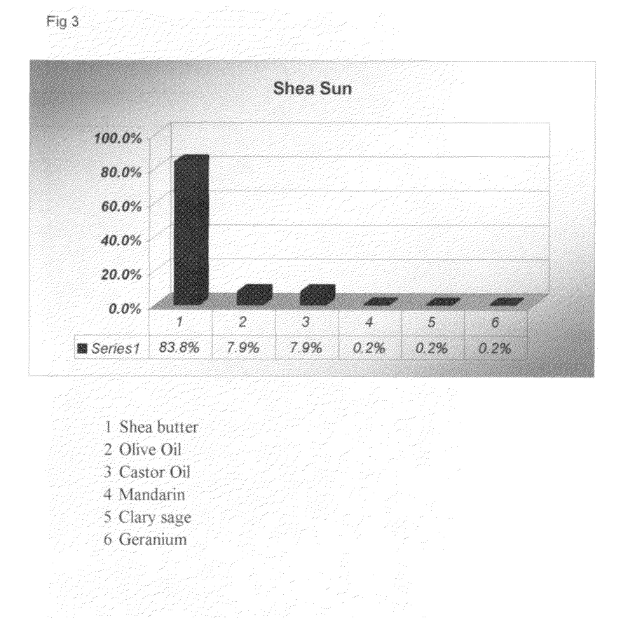

4. The hair and skin formulation according to claim 1 wherein said shea butter is about 84%, olive oil 8%, castor oil 7%, and further comprising about 0.4% by volume of mandarin, 0.3% clary sage and 0.3% geranium. (see FIG. 3).

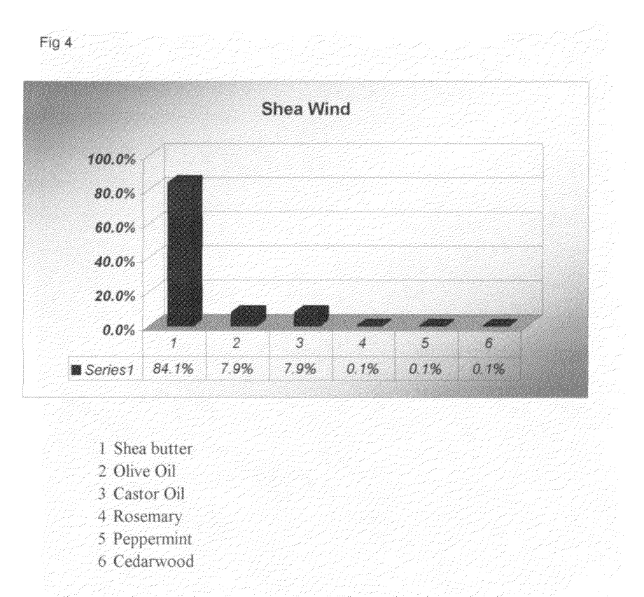

5. The hair and skin formulation according to claim 1 wherein said shea butter is about 84%, olive oil 8%, castor oil 7%, and further comprising about 0.4% by volume of rosemary, 0.3% peppermint, and 0.3% cedarwood. (see FIG. 4).

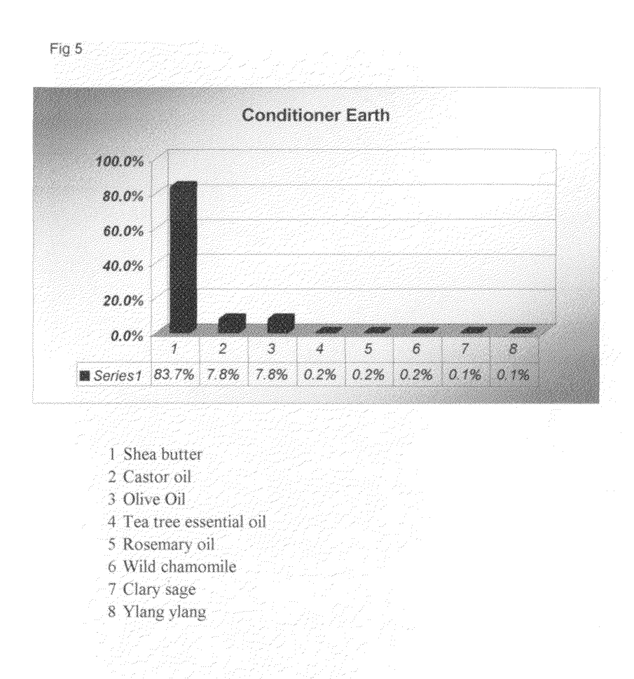

6. The hair and skin formulation according to claim 1 wherein said shea butter is about 83%, olive oil 7%, castor oil 7%, and further comprising about 1% tea tree essential oil, about 1% rosemary oil, about 0.4 wild chamomile, about 0.3% clary sage and about 0.3% of ylang ylang. (see FIG. 5).

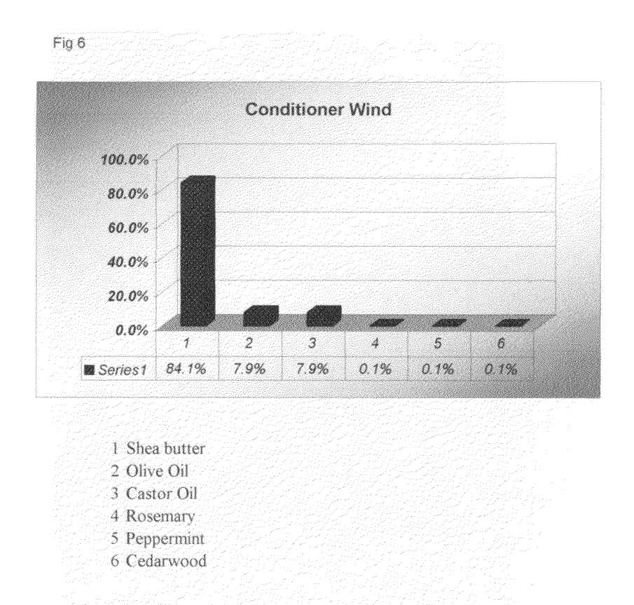

7. The hair and skin formulation according to claim 1 wherein said shea butter is about 83%, olive oil 7%, castor oil 7%, and further comprising about 1% by volume of rosemary, 1% peppermint, and 1% cedarwood. (see FIG. 6).

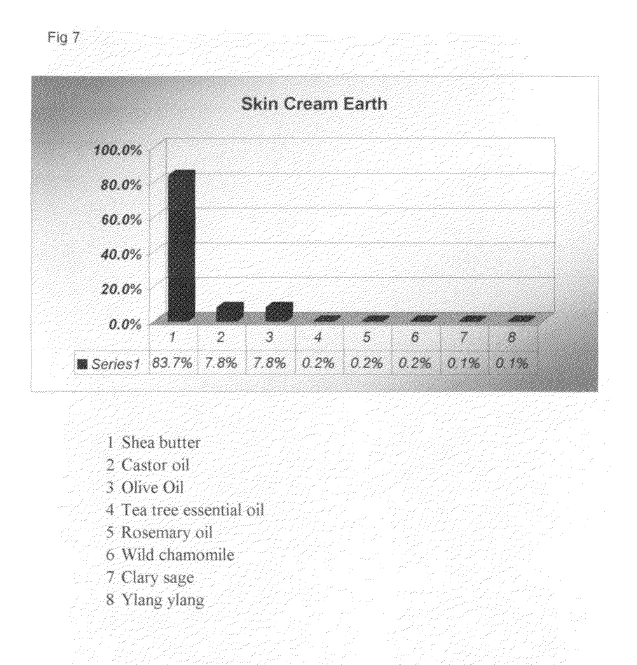

8. The hair and skin formulation according to claim 1 wherein said shea butter is about 83%, olive oil 7%, castor oil 7%, and further comprising about 2% tea tree essential oil, about 0.3% rosemary oil, about 0.3% wild chamomile, about 0.2% clary sage and about 0.2% of ylang ylang. (see FIG. 7).

9. A hair and skin formulation, comprising shea butter, castile soap, olive oil, castor oil, and vegetable glycerin.

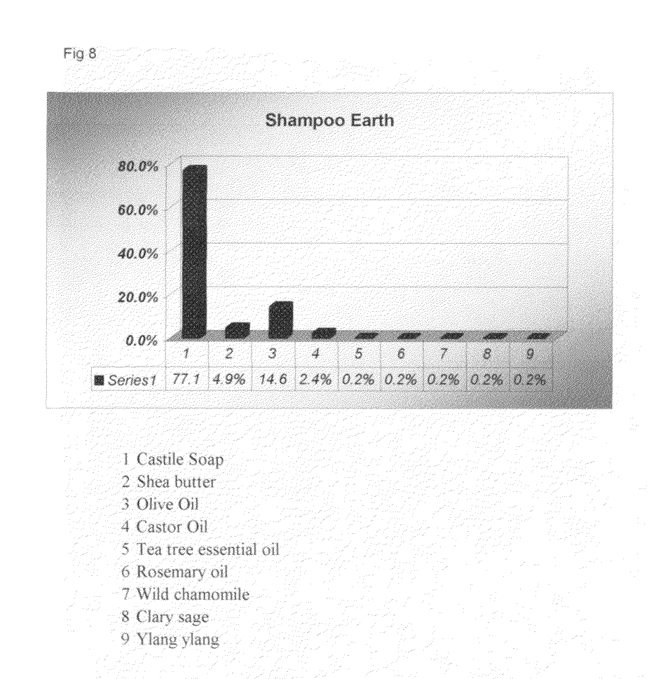

10. The hair and skin formulation according to claim 9 wherein said shea butter is about 5%, castile soap is about 76%, olive oil 7%, castor oil 2%, vegetable glycerin 9% and further comprising about 0.2% tea tree essential oil, about 0.2% rosemary oil, about 0.2% wild chamomile, about 0.2% clary sage and about 0.2% of ylang ylang. (see FIG. 8).

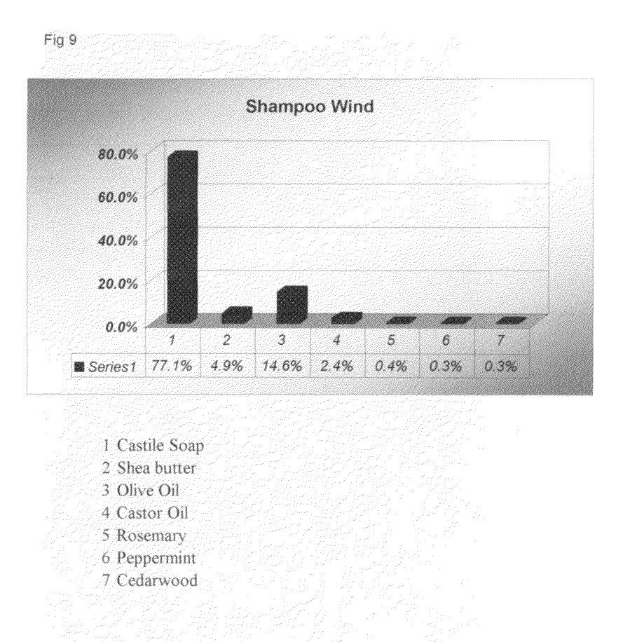

11. The hair and skin formulation according to claim 9 wherein said shea butter is about 5%, castile soap is about 76%, olive oil 7%, castor oil 2%, vegetable glycerin 9% and further comprising about 0.4% by volume of rosemary, 0.3% peppermint, and 0.3% cedarwood. (see FIG. 9).

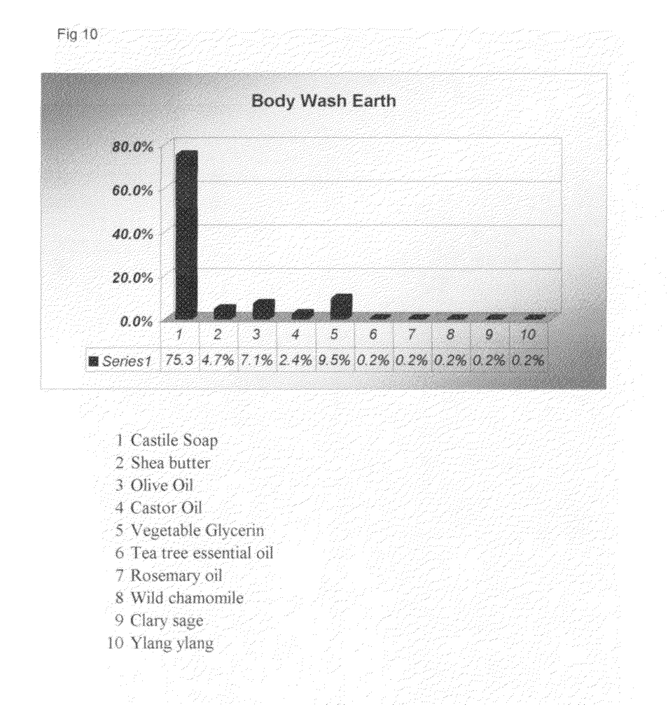

12. The hair and skin formulation according to claim 9 wherein said shea butter is about 5%, castile soap is about 80%, olive oil 7%, castor oil 2%, vegetable glycerin 5% and further comprising about 0.2% tea tree essential oil, about 0.2% rosemary oil, about 0.2% wild chamomile, about 0.2% clary sage and about 0.2% of ylang ylang. (see FIG. 10).

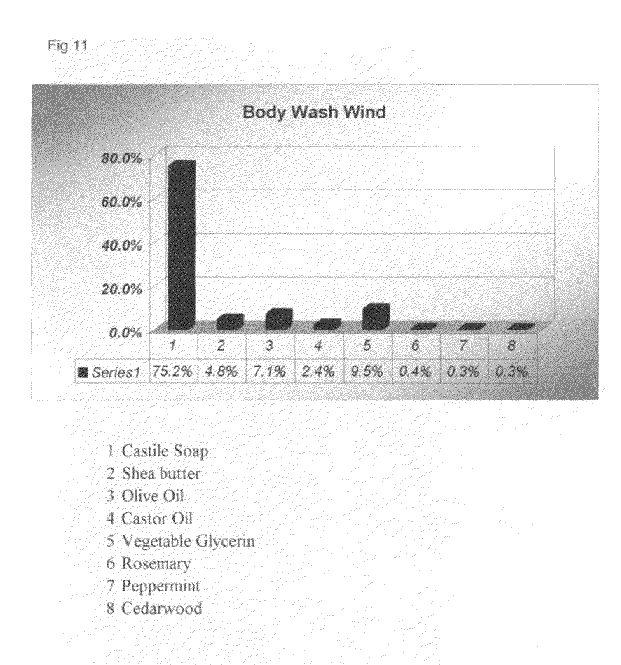

13. The hair and skin formulation according to claim 9 wherein said shea butter is about 5%, castile soap is about 80%, olive oil 7%, castor oil 2%, vegetable glycerin 5% and further comprising about 0.4% by volume of rosemary, 0.3% peppermint, and 0.3% cedarwood. (see FIG. 11).

14. A hair and skin formulation, comprising shea butter, sugarcane, olive oil, and castor oil.

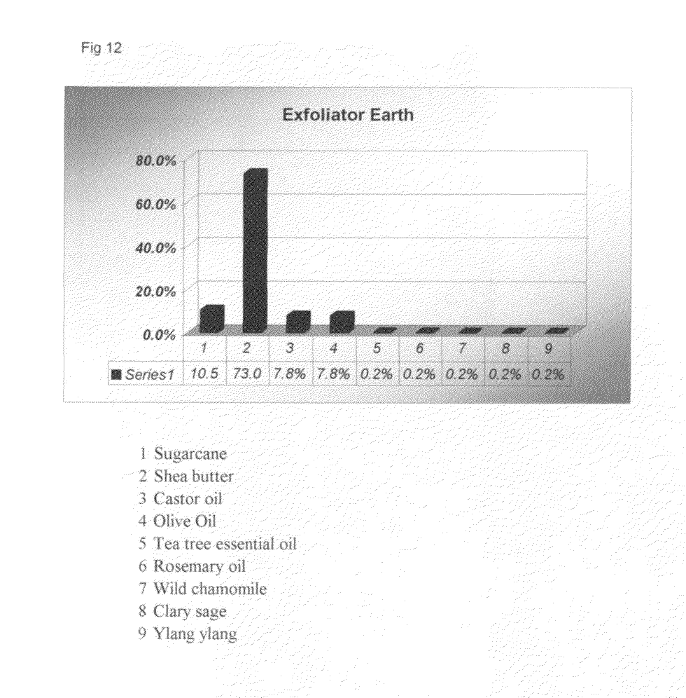

15. The hair and skin formulation according to claim 12 wherein said shea butter is about 73%, olive oil, 10% sugarcane, 8% castor oil 8%, and further comprising about 0.2% tea tree essential oil, about 0.2% rosemary oil, about 0.2% wild chamomile, about 0.2% clary sage and about 0.2% of ylang ylang. (see FIG. 12).

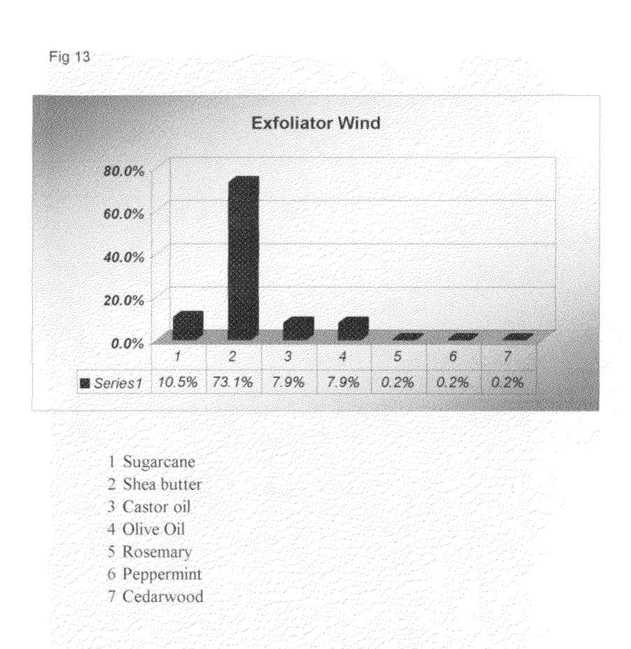

16. The hair and skin formulation according to claim 12 wherein said shea butter is about 73%, olive oil, 10% sugarcane, 8% castor oil 8%, and further comprising about 0.4% by volume of rosemary, 0.3% peppermint, and 0.3% cedarwood. (see FIG. 13).
